(19) United States
(12) Patent Application Publication
(10) Pub. No.: US 2024/0225552 A9
(43) Pub. Date: Jul. 11, 2024

(54) COUPLED PHYSIOLOGICAL SIGNAL MEASURING DEVICE

(71) Applicant: INDUSTRIAL TECHNOLOGY RESEARCH INSTITUTE, Hsinchu (TW)

(72) Inventors: Yun-Yi HUANG, Pingtung City (TW); Yu-Chiao TSAI, Tainan City (TW); Hung-Hsien KO, Zhubei City (TW); Heng-Yin CHEN, Zhubei City (TW)

(73) Assignee: INDUSTRIAL TECHNOLOGY RESEARCH INSTITUTE, Hsinchu (TW)

(21) Appl. No.: 18/099,743

(22) Filed: Jan. 20, 2023

(30) Foreign Application Priority Data
Oct. 24, 2022 (TW) 111140287

(51) Int. Cl. A61B 5/00; A61B 5/304; A61B 5/308; A61B 5/352

(52) U.S. Cl. CPC A61B 5/7203; A61B 5/304; A61B 5/308; A61B 5/352

(57) ABSTRACT

A coupled physiological signal measuring device is provided. The coupled physiological signal measuring device includes at least two measuring electrodes, a signal processing unit and a multiplex feedback circuit unit. The measuring electrodes are used to obtain a real-time physiological signal through measurement. The signal processing unit includes a discharge control element. If an electrostatic surge of the real-time physiological signal meets a condition, a discharge control signal is outputted. The multiplex feedback circuit unit is used to discharge the measuring electrodes according to the discharge control signal.

This application claims the benefit of Taiwan application Serial No. 111140287, filed Oct. 24, 2022, the disclosure of which is incorporated by reference herein in its entirety.

### TECHNICAL FIELD

The disclosure relates in general to a coupled physiological signal measuring device.

### BACKGROUND

Along with the advance in medical and electronic technology, various physiological signal measuring devices are provided. Some physiological signal measuring devices can measure a testee through clothing, which is very convenient to the testee.

Although such type of physiological signal measuring device possesses convenience of use, static electricity and dynamic noises may easily occur and greatly affect judgment accuracy of the physiological signals. Therefore, it has become a prominent task for the industry to provide a solution to the said problem, which has become a bottleneck in the development of relevant technology.

### SUMMARY

The present disclosure relates to a coupled physiological signal measuring device.

According to one embodiment, a coupled physiological signal measuring device is provided. The coupled physiological signal measuring device includes at least two measuring electrodes, a signal processing unit and a multiplex feedback circuit unit. The measuring electrodes are used to obtain a real-time physiological signal through measurement. The signal processing unit includes a discharge control element. If an electrostatic surge of the real-time physiological signal meets a condition, a discharge control signal is outputted. The multiplex feedback circuit unit is used to discharge the measuring electrodes according to the discharge control signal.

According to another embodiment, a coupled physiological signal measuring device is provided. The coupled physiological signal measuring device includes at least two measuring electrodes, at least one ground electrode and a switch. The measuring electrodes are used to obtain a measured physiological signal. The ground electrodes surround the measuring electrodes. The switch is disposed between the measuring electrodes and the ground electrodes.

According to an alternate embodiment, a coupled physiological signal measuring device is provided. The coupled physiological signal measuring device includes at least two measuring electrodes and a signal processing unit. The measuring electrodes are used to obtain a real-time physiological signal through measurement. The signal processing unit includes an active noise cancellation element. The active noise cancellation element is used to perform active noise cancellation on the real-time physiological signal to obtain a real-time denoised physiological signal according to a noise extraction data set. The noise extraction data set is obtained through a machine learning model.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIGS. 1A to 1C are schematic diagrams of a coupled physiological signal measuring device according to an embodiment.

FIG. 2 is a schematic diagram of a coupled physiological signal measuring device performing measurement.

FIG. 3 is a block diagram of a coupled physiological signal measuring device according to an embodiment.

FIG. 4 is a flowchart of an operation method for discharging static electricity using a coupled physiological signal measuring device according to an embodiment.

FIG. 5 illustrates step S120.

FIG. 6 is a schematic diagram of measuring electrodes, ground electrodes and switches according to an embodiment.

FIG. 7 is a schematic diagram of measuring electrodes, ground electrodes and switches according to another embodiment.

FIG. 8 illustrates a diagram of the effect of a static electricity discharging procedure.

FIG. 9 is a block diagram of a coupled physiological signal measuring device according to another embodiment.

FIG. 10 is a flowchart of an operation method for training a machine learning mode and obtaining a noise extraction data set using a coupled physiological signal measuring device according to an embodiment.

FIG. 11 illustrates an operation method of FIG. 10.

FIG. 12 is flowchart of an operation method for active noise cancellation using a coupled physiological signal measuring device according to an embodiment.

FIG. 13 illustrates an operation method of FIG. 12.

FIG. 14 is a block diagram of a coupled physiological signal measuring device according to another embodiment.

FIG. 15 is a flowchart of an operation method for compensating characteristics using a coupled physiological signal measuring device according to an embodiment.

FIG. 16 illustrates an operation method of FIG. 15.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

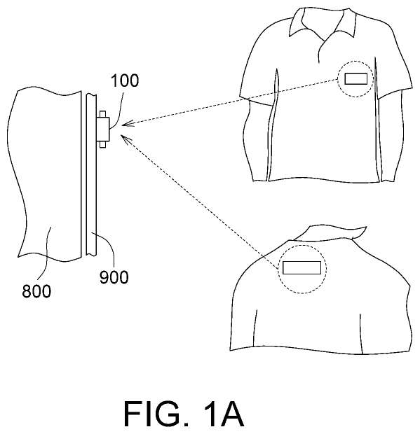

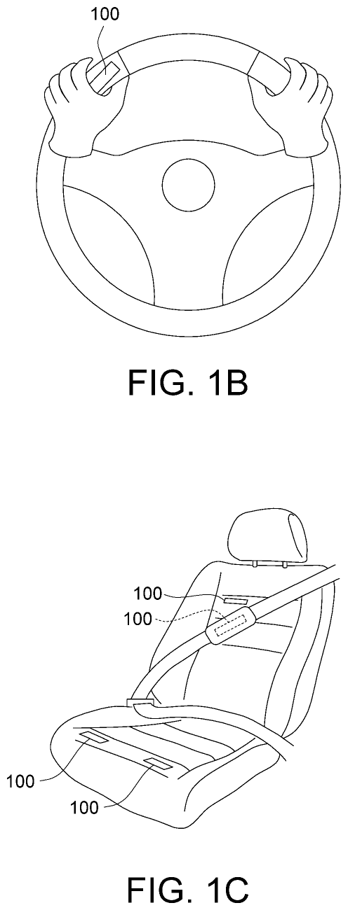

Referring to FIGS. 1A to 1C, schematic diagrams of a coupled physiological signal measuring device 100 according to an embodiment are shown. The coupled physiological signal measuring device 100 is used to measure a testee's physiological signal, such as electrocardiography (ECG) signal, electromyography (EMG) signal or electroencephalography (EEG) signal. The coupled physiological signal measuring device 100 can measure the skin 800 via clothing 900. For instance, the coupled physiological signal measuring device 100 can be arranged on the front chest, the back, the thigh or the arm via the clothing. As indicated in FIG. 1A, the testee can directly attach or fix the coupled physiological signal measuring device 100 on the chest or the back through clothing. As indicated in FIG. 1B, the coupled physiological signal measuring device 100 can be integrated on the steering wheel to measure the skin through plastics, leather or fabrics. As indicated in FIG. 1C, the coupled physiological signal measuring device 100 can also be integrated on a car seat or a seat belt to measure the skin through plastics, leather or fabrics. In other embodiments, the coupled physiological signal measuring device 100 of the present disclosure can also be used in a metaverse world to measure a player's physiological information and then reflect it on the virtual double to increase the interactivity and presence of the game or competition.

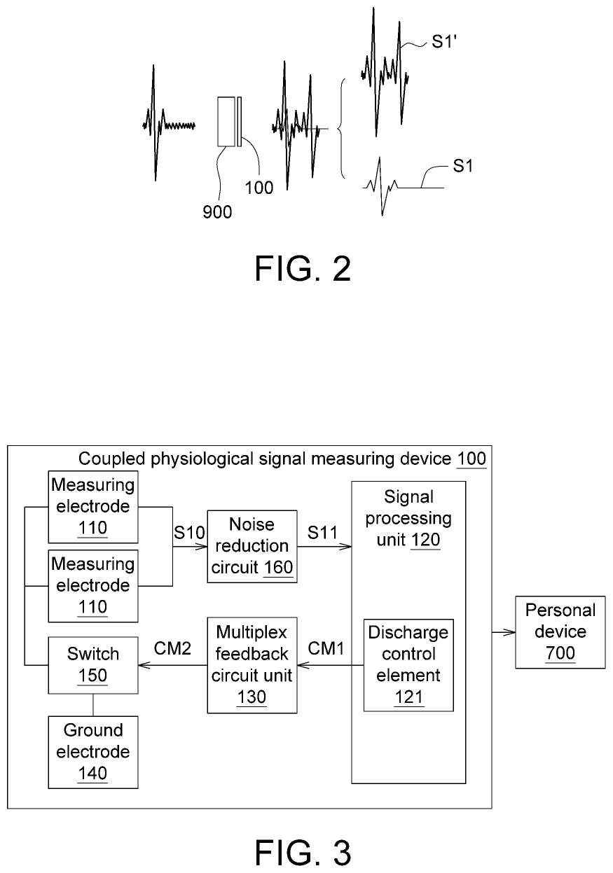

Referring to FIG. 2, a schematic diagram of a coupled physiological signal measuring device 100 performing measurement is shown. The friction of the clothing 900 may easily generate static electricity, and the testee's action may easily cause dynamic noises. Take FIG. 2 for instance. During measurement, what is measured by the coupled physiological signal measuring device 100 includes a noise signal S1′ of static electricity or dynamic noises in addition to a physiological signal S1. As indicated in FIG. 2, the noise signal S1′ significantly affects the interpretation of the physiological signal S1. To avoid the influence of the noise signal S1′, several solutions are provided in the present embodiment.

Referring to FIG. 3, a block diagram of a coupled physiological signal measuring device 100 according to an embodiment is shown. The coupled physiological signal measuring device 100 includes at least two measuring electrodes 110, a signal processing unit 120, a multiplex feedback circuit unit 130, at least one ground electrode 140, a switch 150 and a noise reduction circuit 160. The measuring electrodes 110 and the ground electrodes 140 can be realized by such as a conductive metal pad, a conductive patch, or a conductive cloth. The signal processing unit 120, the multiplex feedback circuit unit 130 and the noise reduction circuit 160 can be used to execute various analysis, processing and control procedures, and can be realized by such as a chip, a circuit board, a circuit, a program code or a storage device for storing program code. The switch 150 is used to conduct or break a circuit, and can be realized by such as a capacitive switch, a resistive switch, a piezoelectric switch, a photo interrupter switch, a magnetic reed switch, a Hall switch, a FET or an MOS. In the coupled physiological signal measuring device 100, the discharge control element 121 of the signal processing unit 120 can determine whether the static electricity is excessive. Once static electricity is determined to be excessive, the discharge control element 121 will execute a static electricity discharging procedure to assure that the measurement of the physiological signal is not affected by static electricity. The coupled physiological signal measuring device 100 is linked to a personal device 700 to transmit the measurement result to the testee or medical personnel. The operations of each element disclosed above are disclosed below with accompanying flowcharts.

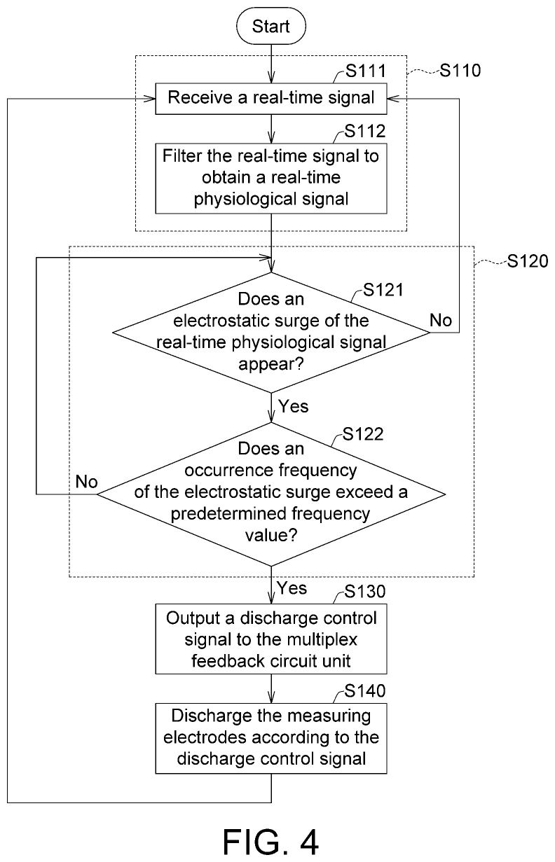

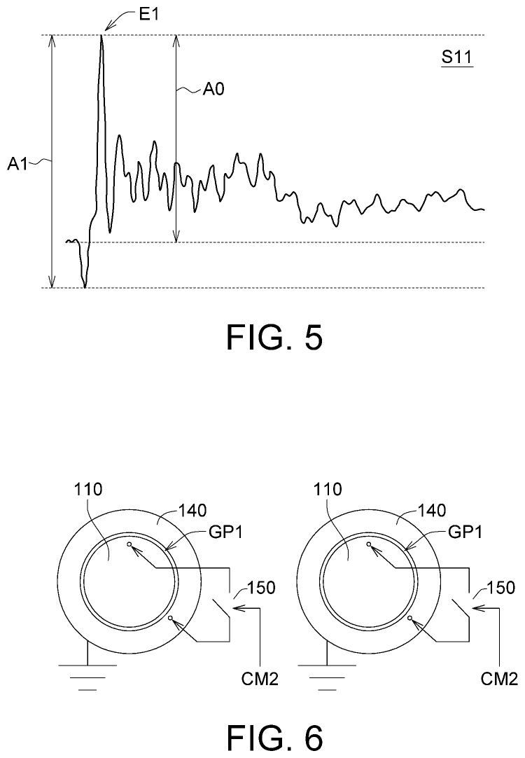

Referring to FIG. 4, a flowchart of an operation method for discharging static electricity using a coupled physiological signal measuring device 100 according to an embodiment is shown. Firstly, the method begins at step S110, a real-time physiological signal S11 is obtained by the measuring electrodes 110 through measurement. Step S110 further includes steps S111 and S112. In step S111, a real-time signal S10 is received by the measuring electrodes 110. In step S112, frequency bands other than the electrocardiography (ECG) signal, the electromyography (EMG) signal or the electroencephalography (EEG) signal are filtered off the real-time signal S10 by the noise reduction circuit 160 to obtain a real-time physiological signal S11. The real-time physiological signal S11 is transmitted to the signal processing unit 120.

Next, the method proceeds to step S120, whether the static electricity judgment value of the real-time physiological signal S11 meets a condition is determined by the signal processing unit 120. If the static electricity judgment value meets the condition, the method proceeds to step S130; if static electricity judgment value does not meet the condition, the method returns to step S110.

Step S120 includes two judgment procedures: step S121 and step S122. Referring to FIG. 5, step S120 is illustrated. In step S121, whether the real-time physiological signal S11 has an electrostatic surge E1 is determined by the discharge control element 121. Take FIG. 5 for instance. The electrostatic surge E1 refers to the part of the real-time physiological signal S11 at which the amplitude A1 exceeds a predetermined amplitude A0. The predetermined amplitude A0 can be exemplified by 1.5V. If the real-time physiological signal S11 has an electrostatic surge E1, the method proceeds to step S122; if the real-time physiological signal S11 does not have an electrostatic surge E1, the method returns to step S111.

In step S122, whether the occurrence frequency of the electrostatic surge E1 exceeds a predetermined frequency value is determined by the discharge control element 121. If the occurrence frequency of the electrostatic surge E1 exceeds predetermined frequency value, the method proceeds to step S130; if the occurrence frequency of the electrostatic surge E1 does not exceed predetermined frequency value, the method returns to step S121. When the occurrence frequency of the electrostatic surge E1 exceeds the predetermined frequency value, this indicates that the static electricity of the measuring electrodes 110 is excessive and needs to be discharged.

In step S130, a discharge control signal CM1 is outputted to the multiplex feedback circuit unit 130 by the discharge control element 121.

Afterwards, the method proceeds to step S140, the measuring electrodes 110 are discharged by the multiplex feedback circuit unit 130 according to the discharge control signal CM1. In the present embodiment, the multiplex feedback circuit unit 130 can complete the static electricity discharging procedure using the ground electrodes 140.

Referring to FIG. 6, a schematic diagram of measuring electrodes 110, ground electrodes 140 and switches 150 according to an embodiment is shown. In the embodiment of FIG. 6, the quantity of the measuring electrodes 110 is two, and the quantity of the ground electrodes 140 is also two. Each ground electrode 140 surrounds a measuring electrode 110. Each measuring electrode 110 is a circular structure, and each ground electrode 140 is an annular structure. The circumstance of each measuring electrode 110 and the circumstance of the corresponding ground electrode 140 are concentric circles. Each measuring electrode 110 and each ground electrode 140 are separated by a gap GP1. The switch 150 is disposed between the measuring electrodes 110 and the ground electrodes 140.

After the multiplex feedback circuit unit 130 receives the discharge control signal CM1, a switch control signal CM2 is inputted to the switch 150 to conduct the path between the measuring electrodes 110 and the ground electrodes 140, so that the measuring electrodes 110 are electrically connected to the ground electrodes 140 and extra static electricity is released.

In an embodiment, the switch control signal CM2 can automatically control the switch 140 to be conducted for a predetermined time, such as 3 seconds. After the predetermined time matures, the switch 150 is automatically turned off, so that the measuring electrodes 110 can resume the measurement function.

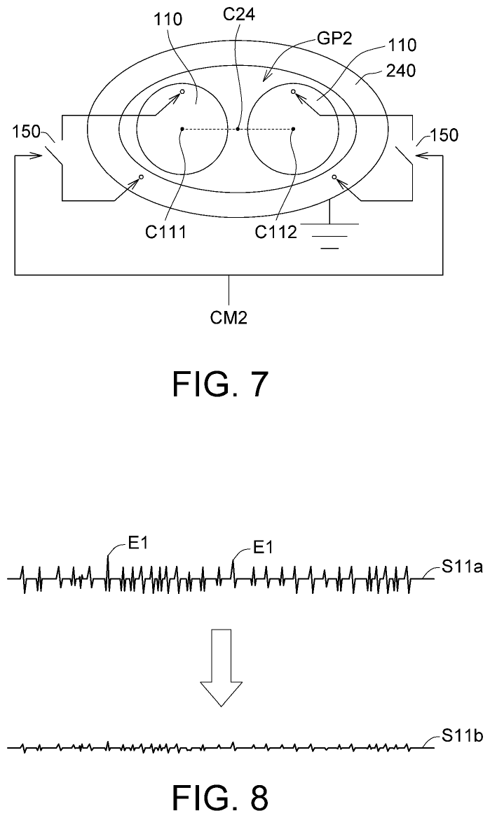

The embodiment of FIG. 6 is applicable to the situation where the measuring electrodes 110 are separated by a larger distance. For instance, the measuring electrodes 110 are used to measure a large muscle group or a large area. Under some circumstances, two measuring electrodes 110 can be adjacently arranged. For instance, the measuring electrodes 110 are used to measure a small muscle group or a small area. Referring to FIG. 7, a schematic diagram of measuring electrodes 110, ground electrodes 240 and switches 150 according to another embodiment is shown. In the embodiment of FIG. 7, the quantity of the measuring electrodes 110 is two, and the quantity of ground electrode 240 is one. The ground electrode 240 surrounds the two measuring electrodes 110. Each measuring electrode 110 is a circular structure, and the ground electrode 240 is an elliptical annular structure. The center C24 of the ground electrode 240 is at the middle point of the connection line connecting the centers C111 and C112 of the measuring electrodes 110. Each measuring electrode 110 and the ground electrode 240 are separated by a gap GP2. The switch 150 is disposed between the measuring electrodes 110 and the ground electrode 240.

After the multiplex feedback circuit unit 130 receives the discharge control signal CM1, a switch control signal CM2 is inputted to the switch 150 to conduct the path between the measuring electrodes 110 and the ground electrode 240, so that the measuring electrodes 110 are electrically connected to the ground electrode 240 and extra static electricity is released.

Referring to FIG. 8, a diagram of the effect of a static electricity discharging procedure is shown. As indicated in FIG. 8, the real-time physiological signal S11a not processed with the static electricity discharging procedure has plenty of electrostatic surges E1, which greatly affect the interpretation of the real-time physiological signal S11a. Conversely, the real-time physiological signal S11b processed with the static electricity discharging procedure is free of electrostatic surges E1, so that accurate interpretation of the real-time physiological signal S11b can be obtained.

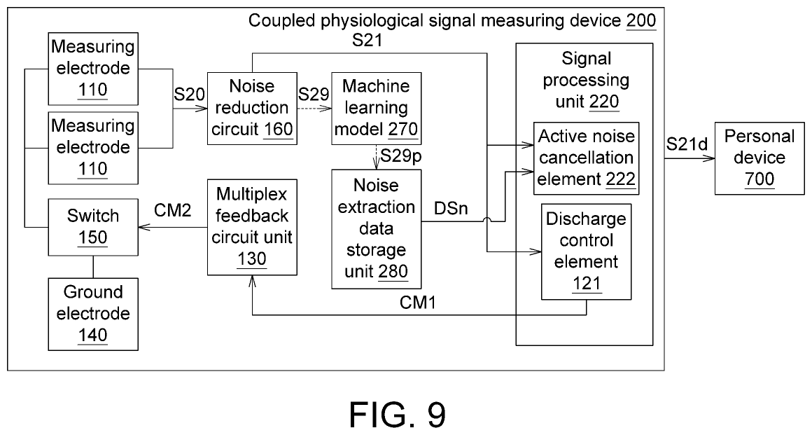

Referring to FIG. 9, a block diagram of a coupled physiological signal measuring device 200 according to another embodiment is shown. The coupled physiological signal measuring device 200 further includes a machine learning model 270 and a noise extraction data storage unit 280, wherein the signal processing unit 220 further includes an active noise cancellation element 222. The machine learning model 270 can be realized by such as a chip, a circuit board, a circuit, a program code or a storage device for storing program code. The noise extraction data storage unit 280 can be realized by such as a memory, a hard disk or a register. The coupled physiological signal measuring device 200 can train the machine learning model 270 through an off-line procedure to obtain a noise extraction data set DSn. The noise extraction data set DSn collects various frequency bands of delicate dynamic noises. The active noise cancellation element 222 can cancel the noises of the real-time physiological signal S21 through the noise extraction data set DSn to avoid the real-time physiological signal S21 being affected by dynamic noises. The operations of each element disclosed above are disclosed below with accompanying flowcharts.

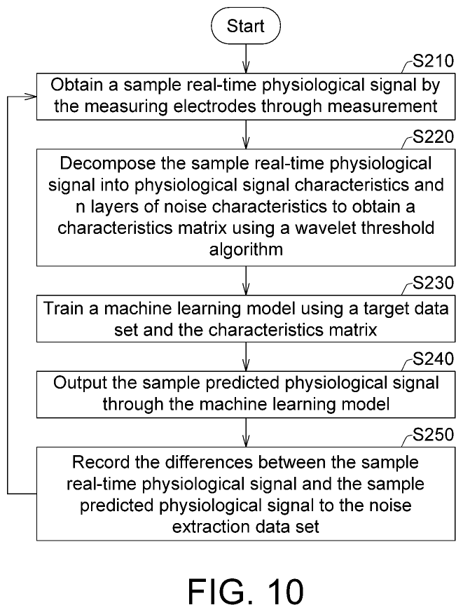

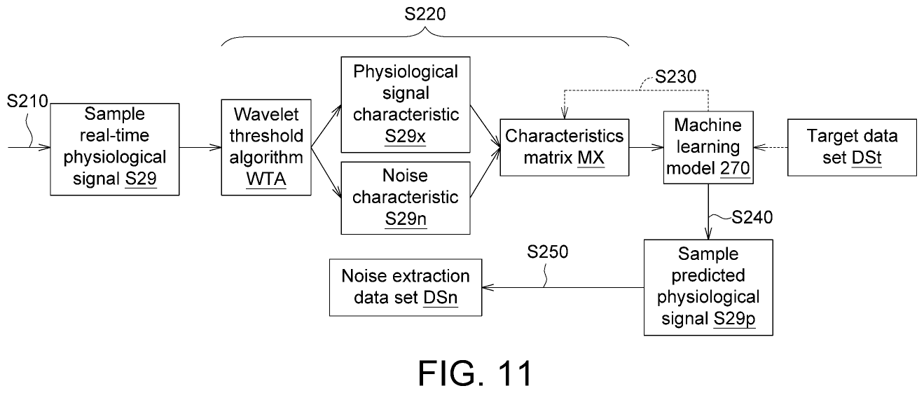

Refer to FIGS. 10 to 11. FIG. 10 is a flowchart of an operation method for training a machine learning mode 270 and obtaining a noise extraction data set DSn using a coupled physiological signal measuring device 200 according to an embodiment. FIG. 11 illustrates the operation method of FIG. 10. In step S210, a sample real-time physiological signal S29 is obtained by the measuring electrodes 110 through measurement. The sample real-time physiological signal S29 can be realized by a signal which has been filtered by the noise reduction circuit 160.

Then, the method proceeds to step S220, the sample real-time physiological signal S29 is decomposed into physiological signal characteristics S29x and n layers of noise characteristics S29n to obtain a characteristics matrix MX using a wavelet threshold algorithm (WTA).

Afterwards, the method proceeds to step S230, a machine learning model 270 is trained using a target data set DSt and the characteristics matrix MX. The target data set DSt is the ground truth of the corresponding sample real-time physiological signal S29 being free of dynamic noises. Steps S210 to S230 can be repeated, so that the machine learning model 270 can be trained using multiple sample real-time physiological signals S29.

Then, the method proceeds to step S240, the sample predicted physiological signal S29p is outputted through the machine learning model 270. The sample predicted physiological signal S29p is obtained through prediction and is free of dynamic noises.

Afterwards, the method proceeds to step S250, the differences between the sample real-time physiological signal S29 and the sample predicted physiological signal S29p are record to the noise extraction data set DSn. In an embodiment, what is recorded to the noise extraction data set DSn is the frequency value corresponding to dynamic noises. After steps S210 to S250 are repeated for several times, multiple frequency values corresponding to the dynamic noises can be recorded to the noise extraction data set DSn.

FIG. 10 and FIG. 11 illustrate the process of training the machine learning model 270 through an off-line procedure to obtain the noise extraction data set DSn. Once the training of the machine learning model 270 is completed and the noise extraction data set DSn is obtained, active noise cancellation can be executed through an on-line procedure.

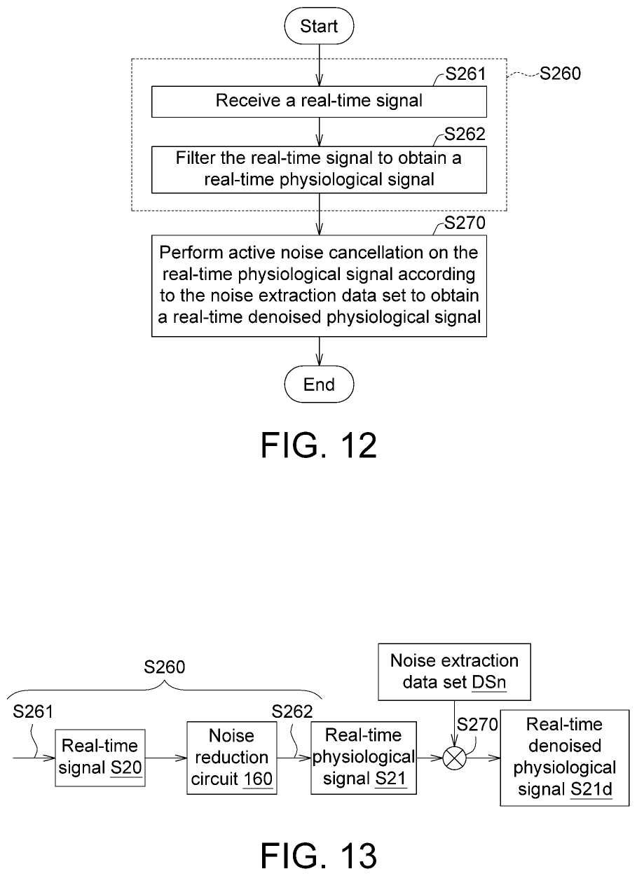

Refer to FIGS. 12 to 13. FIG. 12 is flowchart of an operation method for active noise cancellation using a coupled physiological signal measuring device 200 according to an embodiment. FIG. 13 illustrates an operation method of FIG. 12. In step S260, a real-time physiological signal S21 is obtained by the measuring electrodes 110 through measurement. Step S260 further includes steps S261 and S262. In step S261, a real-time signal S20 is received by the measuring electrodes 110. In step S262, frequency bands other than the electrocardiography (ECG) signal, the electromyography (EMG) signal or the electroencephalography (EEG) signal are filtered off the real-time signal S10 by the noise reduction circuit 160 to obtain a real-time physiological signal S21.

Then, the method proceeds to step S270, active noise cancellation is performed on the real-time physiological signal S21 by the active noise cancellation element 222 according to the noise extraction data set DSn to obtain a real-time denoised physiological signal S21d. The real-time denoised physiological signal S21d is transmitted to a personal device 700 by the coupled physiological signal measuring device 200 (illustrated in FIG. 9). As disclosed above, the noise extraction data set DSn is obtained by the machine learning model 270. In the present step, the active noise cancellation element 222 obtains which frequency bands belong to dynamic noises from the noise extraction data set DSn and filters these frequency bands off the real-time physiological signal S21 to obtain the real-time denoised physiological signal S21d. The active noise cancellation of FIG. 12 and FIG. 13 can effectively avoid the influence of dynamic noises.

Furthermore, the active noise cancellation disclosed above filter off all frequency bands that may possibly have dynamic noises, but in some situations where too many characteristics are removed, the accuracy of signal interpretation will be affected. Here below, solution for signal compensation is provided in an embodiment.

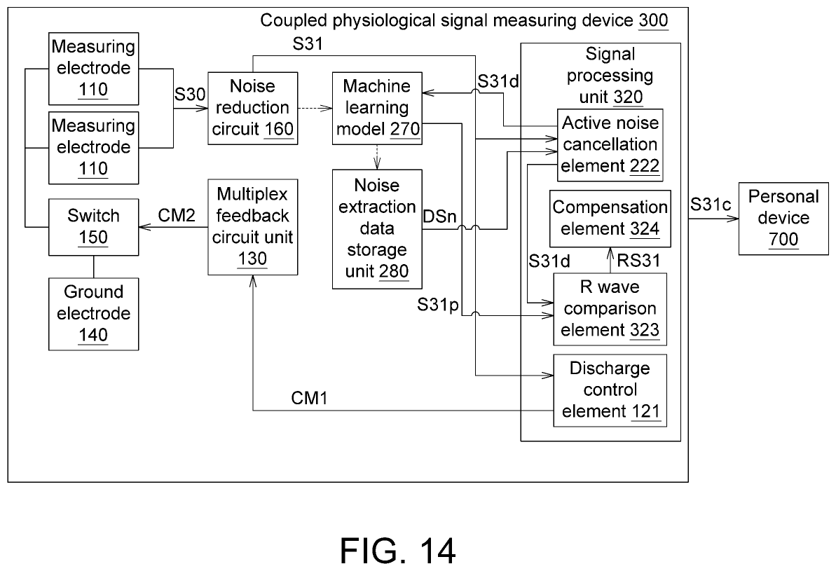

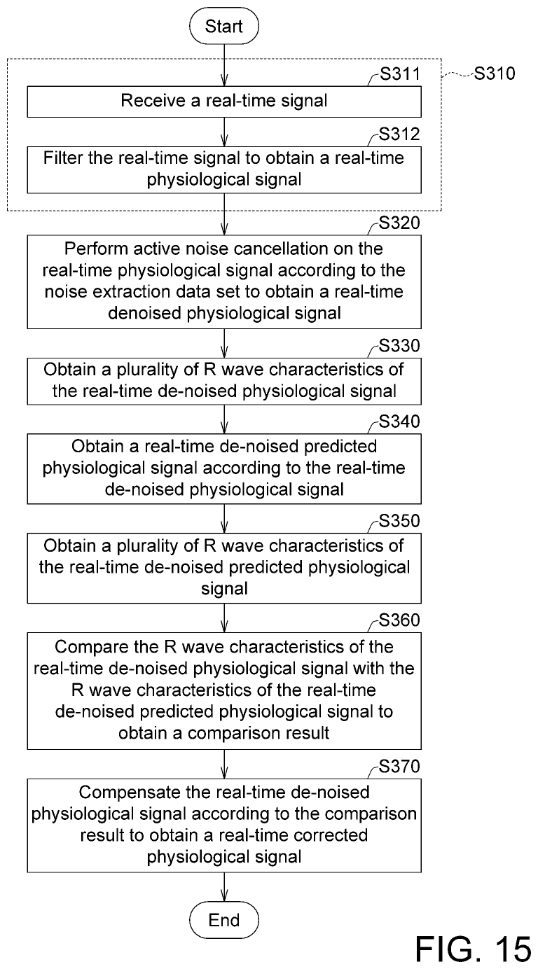

Referring to FIG. 14, a block diagram of a coupled physiological signal measuring device 300 according to another embodiment is shown. In the coupled physiological signal measuring device 300, the signal processing unit 320 further includes an R wave comparison element 323 and a compensation element 324. The R wave comparison element 323 can locate missing characteristics and perform compensation using the compensation element 324 to increase the accuracy of signal interpretation. The operations of each element disclosed above are disclosed below with accompanying flowcharts.

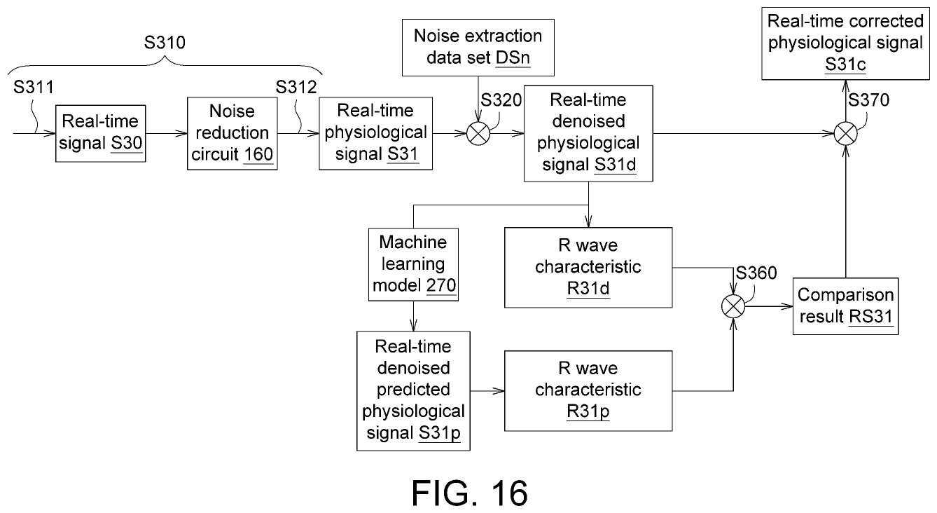

Refer to FIGS. 15 to 16. FIG. 15 is a flowchart of an operation method for compensating characteristics using a coupled physiological signal measuring device 300 according to an embodiment. FIG. 16 illustrates the operation method of FIG. 15. In step S310, a real-time physiological signal S31 is obtained by the measuring electrodes 110 through measurement. Step S310 further includes steps S311 and S312. In step S311, a real-time signal S30 is received by the measuring electrodes 110. In step S312, frequency bands other than the electrocardiography (ECG) signal, the electromyography (EMG) signal or the electroencephalography (EEG) signal are filtered off the real-time signal S30 by the noise reduction circuit 160 to obtain a real-time physiological signal S31.

Then, the method proceeds to step S320, active noise cancellation is performed on the real-time physiological signal S31 by the active noise cancellation element 222 to obtain a real-time denoised physiological signal S31d according to the noise extraction data set DSn. As disclosed above, the noise extraction data set DSn is obtained by the machine learning model 270. In the present step, the active noise cancellation element 222 obtains which frequency bands belong to dynamic noises from the noise extraction data set DSn and filters these frequency bands off the real-time physiological signal S31 to obtain the real-time denoised physiological signal S31d.

Then, the method proceeds to step S330, a plurality of R wave characteristics R31d of the real-time denoised physiological signal S31d are obtained by the R wave comparison element 323 through analysis.

Afterwards, the method proceeds to step S340, a real-time denoised predicted physiological signal S31p is obtained by the machine learning model 270 according to the real-time denoised physiological signal S31d.

Then, the method proceeds to step S350, a plurality of R wave characteristics R31p of the real-time denoised predicted physiological signal S31p are obtained by the R wave comparison element 323 through analysis.

Step S330 and step S350 can be concurrently executed or can exchange their sequence of execution. The real-time denoised physiological signal S31d and the real-time denoised predicted physiological signal S31p have their own advantages and disadvantages.

Then, the method proceeds to step S360, the R wave characteristics R31d of the real-time denoised physiological signal S31d is compared with the R wave characteristics R31p of the real-time denoised predicted physiological signal S31p by the R wave comparison element 323 to obtain a comparison result RS31. Since the real-time denoised physiological signal S31d removes dynamic noises according to the noise extraction data set DSn, more R wave characteristics are removed off the real-time denoised physiological signal S31d. Conversely, more R wave characteristics are maintained in the real-time denoised predicted physiological signal S31p. The comparison result RS31 shows that the R wave characteristics are excessively removed off the real-time denoised physiological signal S31d.

Afterwards, the method proceeds to step S370, the real-time denoised physiological signal S31d is compensated by the compensation element 324 according to the comparison result RS31 to obtain a real-time corrected physiological signal S31c. Then, the real-time corrected physiological signal S31c is transmitted to the personal device 700 through the coupled physiological signal measuring device 300 (illustrated in FIG. 14). In the present step, the compensation element 324 performs gain compensation on the missing R wave characteristics of the real-time denoised physiological signal S31d according to the comparison result RS31.

The waveform of the real-time denoised physiological signal S31d is closer to the original waveform, but too many R wave characteristics are filtered off. Conversely, the waveform of the real-time denoised predicted physiological signal S31p is no longer the original waveform, but the R wave characteristics are not excessively filtered off. Through the comparison procedure and the compensation procedure disclosed above, the advantages of both methods can be maintained and the accuracy in the interpretation of the physiological signal can be greatly increased.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A coupled physiological signal measuring device, comprising:
at least two measuring electrodes, used to obtain a real-time physiological signal through measurement;
a signal processing unit, comprising:
a discharge control element, used to output a discharge control signal if an electrostatic surge of the real-time physiological signal meets a condition; and
a multiplex feedback circuit unit, used to discharge the measuring electrodes according to the discharge control signal.

2. The coupled physiological signal measuring device according to claim 1, wherein an amplitude of the electrostatic surge exceeds a predetermined amplitude.

3. The coupled physiological signal measuring device according to claim 2, wherein the predetermined amplitude is 1.5V.

4. The coupled physiological signal measuring device according to claim 3, wherein the condition is that an occurrence frequency of the electrostatic surge exceeds a predetermined frequency value.

5. The coupled physiological signal measuring device according to claim 1, further comprising:
at least one ground electrode; and
a switch, disposed between the ground electrode and the measuring electrodes;
wherein the multiplex feedback circuit unit controls the switch according to the discharge control signal, so that the measuring electrodes are electrically connected to the ground electrode.

6. The coupled physiological signal measuring device according to claim 5, wherein the measuring electrodes are electrically connected to the ground electrode for a predetermined time.

7. A coupled physiological signal measuring device, comprising:
at least two measuring electrodes, used to obtain a measured physiological signal;
at least one ground electrode, surrounding the measuring electrodes; and
a switch, disposed between the measuring electrodes and the at least one ground electrode.

8. The coupled physiological signal measuring device according to claim 7, wherein a quantity of the at least one ground electrode is two, and each of the ground electrodes surrounds one of the measuring electrodes.

9. The coupled physiological signal measuring device according to claim 8, wherein each of the measuring electrodes is a circular structure, and each of the ground electrodes is an annular structure.

10. The coupled physiological signal measuring device according to claim 9, wherein a circumference of each of the measuring electrodes and a circumstance of one of the ground electrodes are concentric circles.

11. The coupled physiological signal measuring device according to claim 7, wherein a quantity of the at least one ground electrode is one, and the ground electrode surrounds all of the measuring electrodes.

12. The coupled physiological signal measuring device according to claim 11, wherein each of the measuring electrodes is a circular structure, each of the at least one ground electrode is an elliptical annular structure.

13. The coupled physiological signal measuring device according to claim 12, wherein a center of the ground electrode is located at a middle point of a connection line connecting two centers of the measuring electrodes.

14. The coupled physiological signal measuring device according to claim 7, wherein each of the measuring electrodes and the at least one ground electrode are separated by a gap.

15. A coupled physiological signal measuring device, comprising:
at least two measuring electrodes, used to obtain a real-time physiological signal through measurement; and
a signal processing unit, comprising:
an active noise cancellation element, used to perform active noise cancellation on the real-time physiological signal to obtain a real-time denoised physiological signal according to a noise extraction data set, wherein the noise extraction data set is obtained through a machine learning model.

16. The coupled physiological signal measuring device according to claim 15, wherein a plurality of sample physiological signals are inputted to the machine learning model to obtain a plurality of sample predicted physiological signals, and differences between each of the sample physiological signals and each of the sample predicted physiological signals are recorded to the noise extraction data set.

17. The coupled physiological signal measuring device according to claim 16, wherein the noise extraction data set records a plurality of frequency values of a plurality of corresponding dynamic noises.

18. The coupled physiological signal measuring device according to claim 15, wherein the signal processing unit further comprises:
an R wave comparison element, used to compare a plurality of R wave characteristics of the real-time denoised physiological signal with a plurality of R wave characteristics of a real-time denoised predicted physiological signal to obtain a comparison result; and
a compensation element, used to compensate the real-time denoised physiological signal to obtain a real-time corrected physiological signal according to the comparison result.

19. The coupled physiological signal measuring device according to claim 18, wherein the compensation element performs gain compensation on the missing R wave characteristics of the real-time denoised physiological signal according to the comparison result.

20. The coupled physiological signal measuring device according to claim 18, wherein the real-time denoised physiological signal is inputted to the machine learning model to obtain the real-time denoised predicted physiological signal.
